(19) United States
(12) Patent Application Publication
(10) Pub. No.: US 2015/0177114 A1
(43) Pub. Date: Jun. 25, 2015

(54) SYSTEM AND METHOD OF MONITORING PARTICULATE STORAGE

(71) Applicant: AGCO Corporation, Hesston, KS (US)

(72) Inventors: Vijay K. Kapoor, Phoenix, AZ (US); Timothy Dan Buhler, Newton, KS (US); Gerald R. Johnson, Hesston, KS (US)

(21) Appl. No.: 14/575,513

(22) Filed: Dec. 18, 2014

Related U.S. Application Data
(60) Provisional application No. 61/919,206, filed on Dec. 20, 2013.
(60) Provisional application No. 61/919,178, filed on Dec. 20, 2013.

(51) Int. Cl. G01N 15/06; G01B 21/20; G01N 33/00

(52) U.S. Cl. CPC G01N 15/06; G01N 33/0098; G01B 21/20

(57) ABSTRACT

A computing system includes one or more computing devices and a computer-readable medium communicatively coupled with the one or more computing devices. The computer-readable medium has instructions stored thereon which, when executed by the one or more computing devices, cause the one or more computing devices to perform operations including receiving information about a plurality of sensor unit signals, the information including a signal strength associated with each signal at each of a plurality of different locations and sensor data communicated in each of the signals, determining a location of each of the sensor units using the signal strength information, and associating sensor data from each sensor unit with the location of the corresponding sensor unit.

### FIELD

Embodiments of the present invention relate to systems and methods for monitoring conditions in particulate storage areas.

### BACKGROUND

It is often desirable to monitor the conditions of bulk particulate material such as grain, fertilizer or food products, particularly when such particulate material is stored in a container that is subject to variable conditions such as changes in temperature and humidity. If such variable conditions in the particulate storage area can harm the particulate material, monitoring the conditions may be necessary to preserve a safe and stable storage environment.

The above section provides background information related to the present disclosure which is not necessarily prior art.

### SUMMARY

A computing system in accordance with a first embodiment of the invention comprises one or more computing devices and a non-transitory computer-readable medium communicatively coupled with the one or more computing devices. The computer-readable medium has instructions stored thereon which, when executed by the one or more computing devices, cause the one or more computing devices to perform operations comprising receiving information about a plurality of sensor unit signals, the information including a signal strength associated with each signal at each of a plurality of different locations and sensor data communicated in each of the signals, determining a location of each of the sensor units using the signal strength information, and associating sensor data from each sensor unit with the location of the corresponding sensor unit.

A non-transitory computer-readable medium in accordance with another embodiment of the invention has instructions stored thereon which, when executed by one or more computing devices, cause the one or more computing devices to perform operations comprising receiving information about a plurality of sensor unit signals, the information including a signal strength associated with each signal at each of a plurality of different locations and sensor data communicated in each of the signals, determining a location of each of the sensor units relative to the different locations using the signal strength information, and associating sensor data from each sensor unit with the location of the sensor unit.

A computing system in accordance with another embodiment of the invention comprises one or more computing devices, and a non-transitory computer-readable medium communicatively coupled with the one or more computing devices. The computer-readable medium has instructions stored thereon which, when executed by the one or more computing devices, cause the one or more computing devices to perform operations comprising receiving information about a plurality of sensor unit signals, the information including a signal strength associated with each signal at each of a plurality of different locations and sensor data communicated in each of the signals, the sensor data relating to an ambient condition, and determining a location of each of the sensor units relative to the different locations using the signal strength information. The operations further include associating sensor data from each sensor unit with the location of the corresponding sensor unit, using the sensor data from at least one of the plurality of signals to estimate ambient condition values at locations other than the locations of the sensor units, and generating a graphic representation of an area associated with the sensor units and presenting the graphic representation to a user, the graphic representation including indicia of the sensor data associated with each of the sensor unit signals, of the location of the sensor unit associated with each of the sensor unit signals, of the estimated condition values, and of the location associated with each of the estimated condition values.

A computing system in accordance with another embodiment of the invention comprises one or more computing devices and a non-transitory computer-readable medium communicatively coupled with the one or more computing devices and having instructions stored thereon which, when executed by the one or more computing devices, cause the one or more computing devices to perform operations. The operations comprise receiving information about a plurality of sensor unit signals, the information including a signal strength associated with each signal at each of a plurality of different locations, determining a location of each of the sensor units using the signal strength information, and estimating an amount of particulate material associated with the sensor units using the location of each of the sensor units.

These and other important aspects of the present invention are described more fully in the detailed description below. The invention is not limited to the particular methods and systems described herein. Other embodiments may be used and/or changes to the described embodiments may be made without departing from the scope of the claims that follow the detailed description.

### DRAWINGS

Embodiments of the present invention are described in detail below with reference to the attached drawing figures, wherein:

FIG. 1 is a schematic diagram of exemplary computer and communications equipment that may be used to implement certain aspects of the present invention.

FIG. 2 is a block diagram of certain components of a first exemplary particulate monitoring system constructed in accordance with principles of the present invention.

FIG. 3 is a block diagram of certain components of a second exemplary particulate monitoring system constructed in accordance with principles of the present invention.

FIG. 4 is a fragmentary perspective view of a first particulate storage bin configured for use with the system of FIG. 2 or the system of FIG. 3.

FIG. 5 is a fragmentary perspective view of a second particulate storage bin configured for use with the system of FIG. 2 or the system of FIG. 3.

FIG. 6 is a fragmentary perspective view of a truck including a particulate storage area configured for use with the system of FIG. 2 or the system of FIG. 3.

FIG. 7 is a fragmentary perspective view of a railroad car including a particulate storage area configured for use with the system of FIG. 2 or the system of FIG. 3.

FIGS. 8A-8B are cross-sectional views of exemplary wireless interrogators configured for use with the system of FIG. 2 or the system of FIG. 3.

FIGS. 9A-9B are block diagrams of certain components of exemplary wireless interrogators configured for use with the system of FIG. 2 or the system of FIG. 3.

FIGS. 10A-10E are block diagrams of certain components of exemplary sensor units configured for use with the system of FIG. 2 or the system of FIG. 3.

FIG. 11 is a front and side elevation view of a first exemplary housing of a sensor unit configured for use with the system of FIG. 2 or the system of FIG. 3.

FIG. 12 is a front and side elevation view of a second exemplary housing of a sensor unit configured for use with the system of FIG. 2 or the system of FIG. 3.

FIG. 13 is a flow diagram of certain steps performed by a computing system to determine the location of sensor units within a storage area of the system of FIG. 2 or the system of FIG. 3.

FIG. 14 is a first graphical representation of a storage area of the system of FIG. 2 or the system of FIG. 3, the graphical representation including indicia of conditions inside the storage area.

FIG. 15 is a second graphical representation of a storage area of the system of FIG. 2 or the system of FIG. 3, the graphical representation including indicia of conditions inside the storage area.

FIG. 16 is a third graphical representation of a storage area of the system of FIG. 2 or the system of FIG. 3, the graphical representation including indicia of conditions inside the storage area.

FIG. 17 is a fourth graphical representation of a storage area of the system of FIG. 2 or the system of FIG. 3, the graphical representation including indicia of conditions inside the storage area.

The drawing figures do not limit the present invention to the specific embodiments disclosed and described herein. The drawings are not necessarily to scale, emphasis instead being placed upon clearly illustrating the principles of the invention.

### DESCRIPTION

The following detailed description of embodiments of the invention references the accompanying drawings. The embodiments are intended to describe aspects of the invention in sufficient detail to enable those skilled in the art to practice the invention. Other embodiments can be utilized and changes can be made without departing from the scope of the claims. The following description is, therefore, not to be taken in a limiting sense.

In this description, references to “one embodiment”, “an embodiment”, or “embodiments” mean that the feature or features being referred to are included in at least one embodiment of the technology. Separate references to “one embodiment”, “an embodiment”, or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments, but is not necessarily included. Thus, the present technology can include a variety of combinations and/or integrations of the embodiments described herein.

Embodiments of the present invention relate to systems and methods of assessing and monitoring one or more conditions within a particulate storage area to, for example, detect and respond to the occurrence of an adverse condition affecting the storage area. More particularly, embodiments of the invention involve the use of sensor units distributed throughout particulate material in the storage area, the sensor units being configured to sense one or more conditions and to communicate wirelessly with a plurality of wireless interrogators associated with the storage area to thereby communicate sensor data to the interrogators. One or more computing devices determine a position of each of the sensor units using, for example, triangulation, and associate the sensor data from each sensor unit with the location of the sensor unit. The condition and position data may then be used to generate alerts to users, to activate machines or systems to address adverse conditions, and/or to present profile information to users.

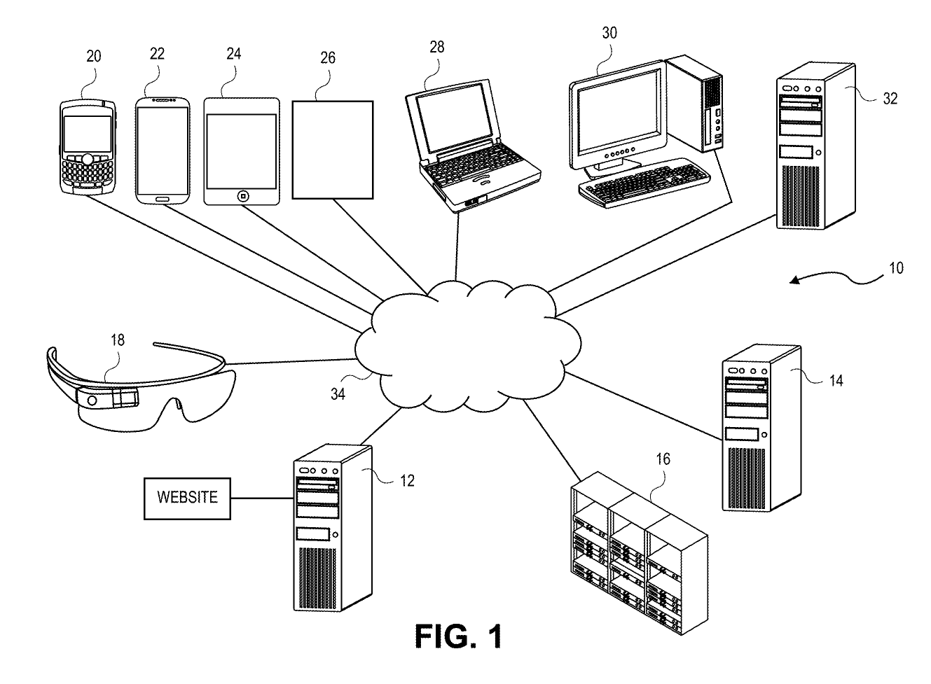

Certain aspects of the present invention can be implemented by, or with the assistance of, computing equipment such as computers and associated devices including data storage devices. Such aspects of the invention may be implemented in hardware, software, firmware, or a combination thereof. In one exemplary embodiment, aspects of the invention are implemented with a computer program or programs that operate computer and communications equipment broadly referred to by the reference numeral 10 in FIG. 1. The exemplary computer and communications equipment 10 may include one or more host computers or systems 12, 14, 16 (hereinafter referred to simply as “host computers”) and a plurality of electronic or computing devices 18, 20, 22, 24, 26, 28, 30, 32 that may access the host computers via a communications network 34. The computer programs and equipment illustrated and described herein are merely examples of programs and equipment that may be used to implement aspects of the invention and may be replaced with other programs and computer equipment without departing from the scope of the invention.

The host computers 12, 14, 16 may serve as repositories for data and programs used to implement certain aspects of the present invention as described in more detail below. The host computers 12, 14, 16 may be any computing and/or data storage devices such as network or server computers and may be connected to a firewall to prevent tampering with information stored on or accessible by the computers.

One of the host computers, such as host computer 12, may be a device that operates or hosts a website accessible by at least some of the devices 18-32. The host computer 12 may include conventional web hosting operating software and an Internet connection, and is assigned a URL and corresponding domain name so that the website hosted thereon can be accessed via the Internet in a conventional manner. One or more of the host computers 12, 14, 16 may host and support a database for storing GNSS information, as explained below. The database may be accessible, for example, via the website operated by the host computer 12.

Although three host computers 12, 14, 16 are described and illustrated herein, embodiments of the invention may use any combination of host computers and/or other computers or equipment. For example, the computer-implemented features and services described herein may be divided between the host computers 12, 14, 16 or may all be implemented with only one of the host computers. Furthermore, the functionality of the host computers 12, 14, 16 may be distributed amongst many different computers in a cloud computing environment.

The electronic devices 18-32 may include various types of devices that can access the host computers 12, 14, 16 via the communications network 34. By way of example, the electronic devices 18-32 may include one or more laptop, personal or network computers 28-32 as well as one or more smart phones, tablet computing devices or other handheld, wearable and/or personal computing devices 18-24. The devices 18-32 may include one or more devices or systems 26 embedded in or otherwise associated with a particulate storage area wherein the device or system 26 enables an electronic device or system associated with the storage area, a user, or both to access one or more of the host computers 12, 14, 16. Each of the electronic devices 18-32 may include or be able to access a web browser and a conventional Internet connection such as a wired or wireless data connection. As explained below, the device or system 26 may be associated with one or more components of a particulate monitoring system and may be operable to communicate with one or more of the electronic devices 12-24, 28-32 to communicate data and information collected from a particulate storage area to the one or more of the electronic devices 12-24, 28-32 and/or to receive instructions from the one or more of the electronic devices 12-24, 28-32.

The communications network 34 preferably is or includes the Internet but may also include other communications networks such as a local area network, a wide area network, a wireless network, or an intranet. The communications network 34 may also be a combination of several networks. For example, the electronic devices 18-32 may wirelessly communicate with a computer or hub in a store via a local area network (e.g., a Wi-Fi network), which in turn communicates with one or more of the host computers 12, 14, 16 via the Internet or other communication network.

One or more computer programs implementing certain aspects of the present invention may be stored in or on computer-readable media residing on or accessible by the computing and communications equipment 10. The one or more computer programs preferably comprise ordered listings of executable instructions for implementing logical functions in the host computers 12, 14, 16 and/or the devices 18-32. The one or more computer programs can be embodied in any computer-readable medium for use by or in connection with an instruction execution system, apparatus, or device, such as a computer-based system, processor-containing system, or other system that can fetch the instructions from the instruction execution system, apparatus, or device, and execute the instructions. In the context of this application, a “computer-readable medium” can be any means that can contain, store, communicate, propagate or transport the program for use by or in connection with the instruction execution system, apparatus, or device. The computer-readable medium can be, for example, but not limited to, an electronic, magnetic, optical, electromagnetic, infrared, or semi-conductor system, apparatus, device, or propagation medium. More specific, although not inclusive, examples of the computer-readable medium would include the following: an electrical connection having one or more wires, a portable computer diskette, a random access memory (RAM), a read-only memory (ROM), an erasable, programmable, read-only memory (EPROM or Flash memory), an optical fiber, and a portable compact disk read-only memory (CDROM).

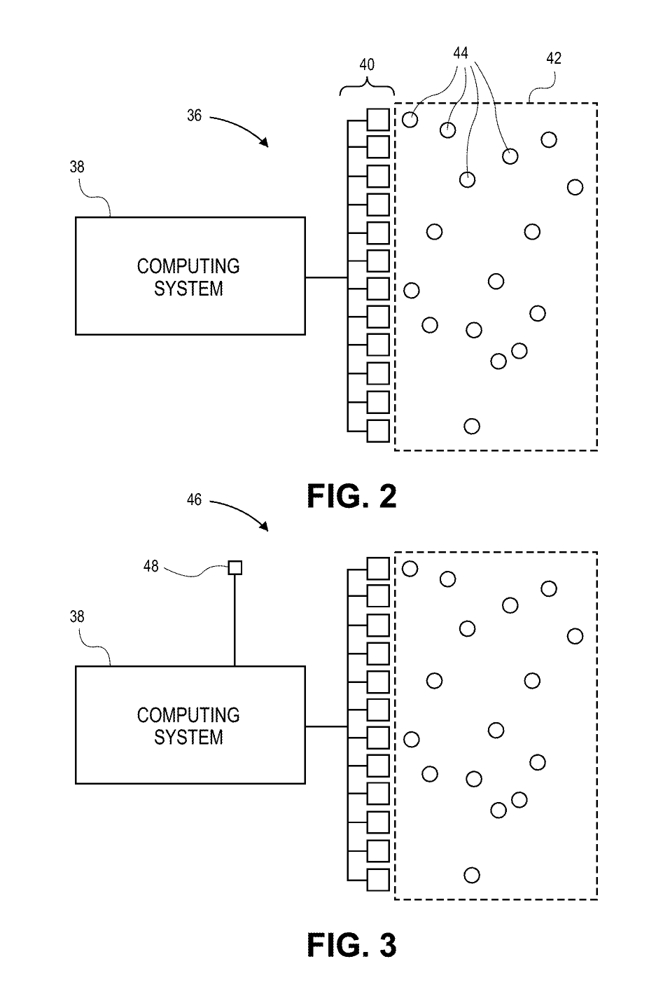

An exemplary system 36 in accordance with embodiments of the invention is illustrated in FIG. 2. The system 36 broadly includes a computing system 38, a plurality of interrogators 40 associated with a storage area 42 and a plurality of sensor units 44 in the storage area. Another exemplary system 46 in accordance with embodiments of the invention is illustrated in FIG. 3, the system 46 further including a sensor 48 in communication with the computing system 38. The sensor 48 may be positioned in the same region or vicinity as the storage area 42 to collect ambient condition information and communicate the information to the computing system 38.

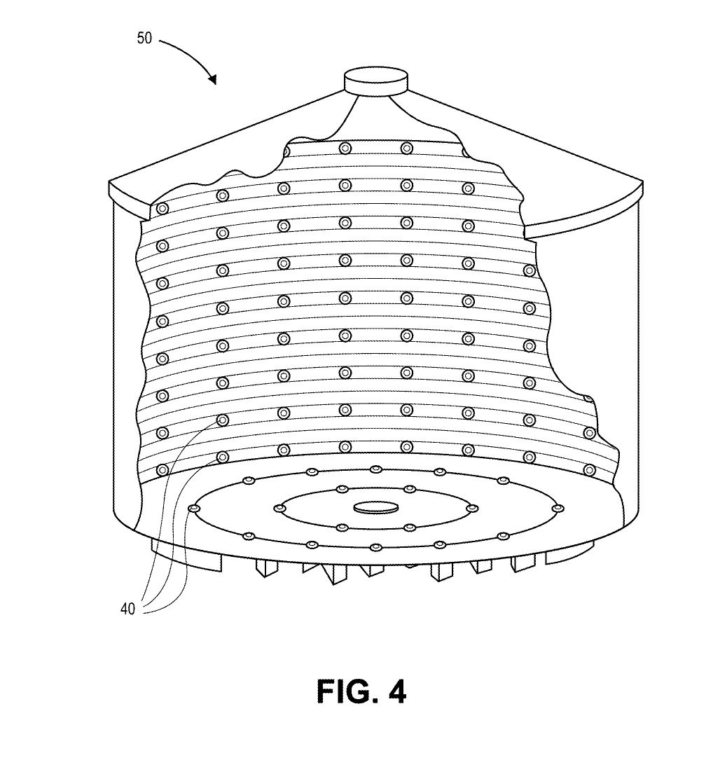

FIGS. 4 through 7 illustrate various exemplary particulate storage areas 50, 52, 54, 56 suitable for use with the present invention, including stationary storage bins 50 and 52 and mobile storage units 54 and 56. While the illustrated particulate storage areas are adapted for storing grain or other agricultural material including, for example, fertilizer or processed animal feed, the invention is not so limited. Virtually any particulate storage area of any size and adapted for use with any particulate material may be used with the invention. By way of example, the present invention may also be used with a storage container adapted for use in the food production industry to hold flour, sugar or other cooking materials.

The particulate material may be fine, such as grain with small kernels or a powder such as flour, or may be coarse, such as ears of corn or other agricultural material larger than grain. The particulate storage area may be of virtually any size and shape, whether inside or outside, open or closed, conventional or non-conventional in nature. The storage area may be, for example, as simple as a flat surface on which particulate material is heaped.

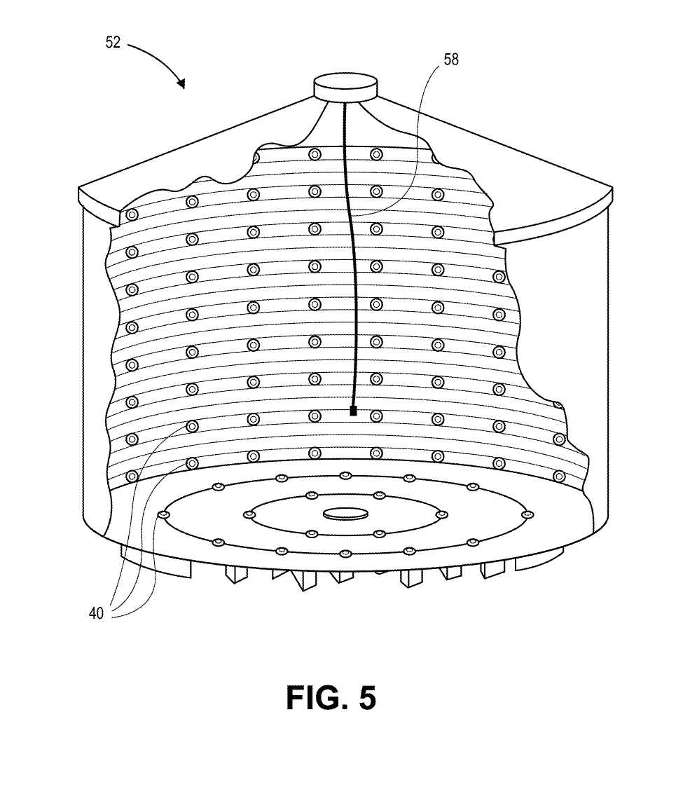

A plurality of wireless interrogators 40 are associated with the particulate storage area 42 for communication with sensor units 44 inside the storage area 42, as explained below in greater detail. As illustrated in FIG. 4, the interrogators 40 may be placed on an inside surface of a grain storage bin 50 or otherwise supported by a wall or other structural element of the storage area 42. The interrogators 40 may be placed outside or inside the storage area 42, such as on a cable 58 or other device suspended within a grain storage bin 52, as illustrated in FIG. 5. The interrogators 40 include electrical components, including wireless transmitters and/or receivers, therefore their placement on, near or in a storage area structure may need to accommodate wires, power sources or other components associated with the interrogators 40. By way of example, placing the interrogators 40 outside of an enclosed metal storage area may limit or prevent wireless communications between the interrogators and sensor units inside the storage area.

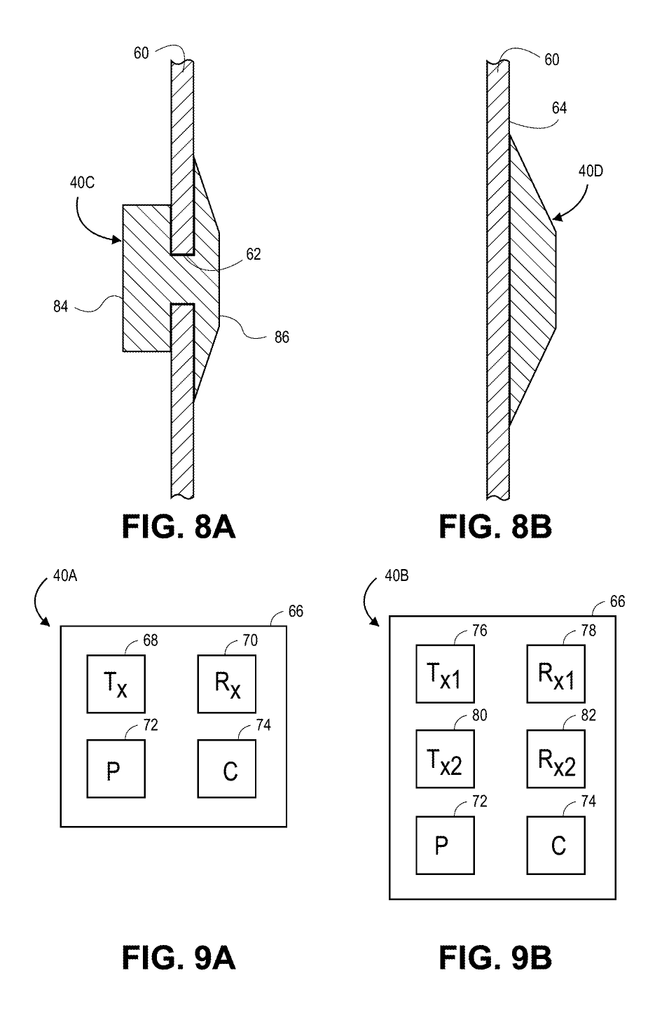

The storage area 42 may be especially designed or modified to accommodate the interrogators 40 and any components used with the interrogators 40. As illustrated in FIG. 8A, for example, a wall 60 of a storage area may include one or more apertures 62 for receiving and supporting the interrogators 40. Alternatively, each interrogator 40 may be secured to an inner surface 64 of a wall of the storage area with, for example, an adhesive or a magnetic, as illustrated in FIG. 8B.

The computing system 38 may be configured to monitor and track conditions in more than one particulate storage area. By way of example, the computing system 38 may be configured to monitor and track the conditions in a grain tank of a combine harvester, in a stationary grain storage bin and in a transport vehicle, such as a grain cart, grain truck or hopper railcar. Thus, each of multiple different storage areas may be connected to or may be otherwise associated with the computing system 38 such that the computing system 38 can monitor and record conditions in all of the storage areas. The interrogators 40 may be configured to communicate identification information to the computing system 38, wherein the identification information is unique to the storage area 42 and enables the computing system 38 to associate data and information collected from the interrogators with the storage area 42. If multiple storage areas are associated with a batch of particulate material, the computing system 38 may be configured to track the conditions of the particulate material as it is transferred from one storage area to another.

The wireless interrogators 40 are configured and positioned to communicate with at least some of the sensor units 44 positioned inside the storage area 42. Thus, each of the wireless interrogators 40 is configured to generate a wireless signal that is directed, at least partially, toward sensor units 44 inside the storage area 42. The interrogators 40 may include components and functionality that is similar to that of radio frequency identification (RFID) interrogators configured to generate RF signals that energize RFID tags and to receive RF signals transmitted by the RFID tags. While the present invention is not limited to identification, each of the sensor units 44 may be configured similar to an RFID tag and operable to transmit a signal carrying sensor data when energized by an RF signal emitted by one or more of the interrogators 40.

As illustrated in FIGS. 4-7, the interrogators 40 may be placed to form a grid or array in or near the storage area 42. The locations of the interrogators 40 are used to triangulate the positions of the sensor units 44, as explained below. Thus, the interrogators 40 are used to collect data for determining a position of each of the sensor units 44, including signal strength and sensor unit identification data, and for receiving sensor data communicated by the sensor units 44 in the wireless signals. The computing system 38 uses all of this data to assess conditions inside the storage area 42, including inside a heap or mass of particulate material in the storage area 42.

The interrogators 40 are preferably positioned to maximize individual and/or collective communication with the sensor units 44. Thus, the optimal position of the interrogators 40 will vary from one implementation to another according to such factors as the size and shape of the storage area 42, the number of interrogators 40, the range of the wireless components of the interrogators 40 and the sensor units 44, and the type of particulate material inside the particulate storage area 42. Regardless of how the interrogators 40 are positioned, the location of each may be stored in (or communicated to) the computing system 38 to enable the computing system 38 to determine the locations of the sensor units 44 based on the locations of the interrogators 40, as explained below in greater detail. Thus, the size and shape of the particulate storage area 42, as well as the location of each of the interrogators 40 relative to the storage area 42, may be loaded or programmed into the computing system 38, and the computing system 38 may generate a virtual model of the storage area 42 with indicia of both the sensor data and the location of the sensor data associated with each of the sensor units 44.

A block diagram of various components of an exemplary interrogator 40A constructed in accordance with a first embodiment is illustrated in FIG. 9A. The interrogator 40A includes a housing 66, a wireless transmitter 68 (e.g., an RF transmitter), a wireless receiver 70 (e.g., an RF receiver), a power source 72, and communications and control circuitry 74. The transmitter 68 is configured to communicate wireless signals to the sensor units 44 to transfer data to the sensor units and/or to energize the sensor units 44. If the sensor units 44 are active (that is, have their own power source), the transmitter 68 may only communicate a request for transmission, wherein the sensor units 44 respond by transmitting a wireless signal detected by the wireless receiver 70. If the sensor units 44 are passive (that is, do not have their own power source), the transmitter 68 may communicate a wireless signal configured to energize one or more sensor units 44 within range of the transmitter 68, wherein the energized sensor unit 44 transmits a wireless signal detected by the wireless receiver 70. The transmitter 68 must be capable of transmitting a signal that penetrates particulate matter stored in the storage area 42 to reach—and communicate with—at least some sensor units 44 mixed with the particulate material inside the storage area 42. In some implementations of the invention the storage area 42 may be large, wherein the transmitters must communicate over distances of several meters up to, for example, ten or twenty meters.

The power source 72 may be an internal power source, such as a battery, or may be a connection to an external source of power, such as a connector configured to connect to a cable or wire providing electrical power. The communications and control circuitry 74 direct operation of the interrogator 40A and may enable communications between two or more interrogators, between an interrogator and the computing system 38, or both. The communications and control circuitry 74 may include one or more processing circuits or devices, such as microcontrollers or microprocessors, and may include components and logic for determining a strength of a received signal by, for example, measuring an amount of energy in the received signal.

A block diagram of various components of another exemplary interrogator 40B constructed in accordance with a second embodiment is illustrated in FIG. 9B. The configuration of the interrogator 40B is similar to that of the interrogator 40A, except that the interrogator 40B includes first 76 and second 80 transmitters and first 78 and second 82 receivers. This configuration may be useful to, for example, enable communications between multiple interrogators 40, between interrogators 40 and the computing system 38, or both. The first transmitter 76 and first receiver 78 may be configured to communicate with sensor units 44 inside the particulate storage area 42, while the second transmitter 80 and second receiver 82 may be configured to communicate with other devices or systems, such as other interrogators, the computing system 38, or both.

As mentioned above, the wireless interrogators 40 may be positioned external to (but near) the particulate storage area 42, on a structure defining or otherwise associated with the storage area 42, or inside the storage area 42. An exemplary interrogator 40C is illustrated in FIG. 8A. The interrogator 40C is mounted on a wall 60 of the storage area 42 such that a first, inner portion 84 of the interrogator 40C is on an outside of the storage area 42 and a second, outer portion 86 of the interrogator 40C is on an inside of the storage area 42. A transmitter and a receiver associated with the interrogator 40C may correspond to the inner portion 86 of the interrogator, for example, while the remaining components may correspond to the outer portion 84. In this configuration, wires used for enabling communications, for delivering power, or both may be attached to the outer portion 84 of the interrogator 40C, thus eliminating the need to place wires inside the storage area 42.

Another interrogator configuration 40D is illustrated in FIG. 8B. The interrogator 40D is configured to attach to an inner surface 64 of the storage area 42 without the need for an aperture in the wall 60. The interrogator 40D may contain a power source, such as a battery, to eliminate the need for wires or cables to deliver power. Furthermore, all communications may be performed wirelessly, thereby eliminating the need to interconnect the interrogator 40D with wires or cables to enable communications. Both of the interrogator designs 40C and 40D include housings with tapered edges that define a smooth or substantially smooth transition between an inner surface (e.g., 64) of the storage area and the interrogator housing. This may be desirable, for example, to facilitate movement of particulate material along the interrogator housing and to prevent build-up of particulate material on the interrogator housing.

The interrogator 40D may be secured to the surface 64 using, for example, a magnet or an adhesive. The interrogators 40C, 40D may be attached to walls of the storage area 42, such as outer walls defining a perimeter of the storage area 42, or may be attached to other structures associated with the storage area 42. A cable or other structure may be suspended or otherwise positioned inside the particulate storage area and configured to hold one or more of the interrogators, as illustrated in FIG. 5. Positioning one or more interrogators 40 inside the storage area, as opposed to only at or near a perimeter of the storage area, may present the advantage of increasing the interrogator range, the accuracy of the data collected by the interrogators, or both.

The interrogator housing 66 may vary substantially in shape and size from one embodiment of the invention to another. By way of example, an outer diameter of each interrogator 40 may be between about 1.0 cm and about 20 cm, and more particularly between about 2.0 cm and about 10 cm. The height of the inner portion 86 of interrogator 40C or the total height of the interrogator 40D may be between about 1.0 mm and about 2.0 cm and, more particularly, between about 2.0 mm and about 1.0 cm.

As mentioned above, the interrogators 40 are configured to communicate data or information to an external device or system, such as a computing system 38, to enable further processing, distribution or other uses of the data or information. Some or all of the interrogators 40 may be communicatively interconnected in a mesh network or other network topology wherein a subset of the interrogators 40 (perhaps just one) is in communication with the computing system 38. Alternatively, each of the interrogators 40 may be communicatively coupled with the computing system 38. In some embodiments, the device or system 26 illustrated in FIG. 1 may include, or may be in communication with, one of the interrogators 40 to enable communications with any of the other devices 12-24, 28-32.

The sensor units 44 are positioned inside the particulate storage area 42 during operation and are configured to communicate condition information to the interrogators 40. The sensor units 44 may be distributed throughout at least a portion of particulate material in the storage area 42 to collect and communicate condition information relating to the storage area and/or the particulate material. Thus, each sensor unit 44 is a self-contained device including one or more sensors and one or more wireless communications components for enabling wireless communications with the interrogators 40.

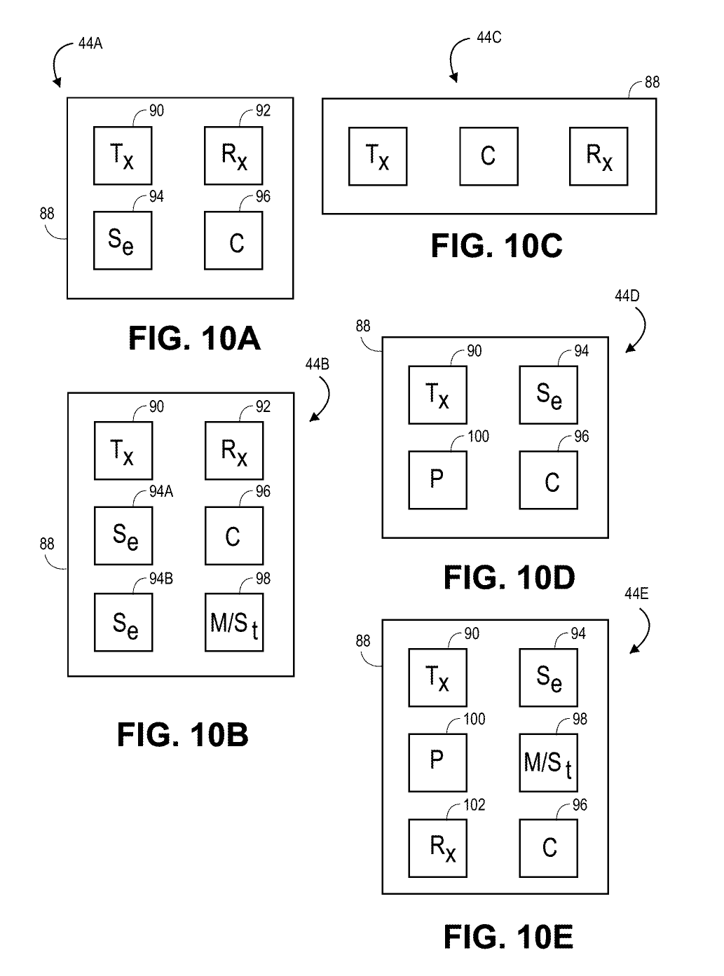

FIGS. 10A through 10E illustrate block diagrams of exemplary embodiments of the sensor units. In a first exemplary embodiment, illustrated in FIG. 10A, a sensor unit 44A is configured for passive operation and derives power from wireless signals generated by one or more of the interrogators 40, in a manner similar to the operation of an RFID tag, as explained above. More particularly, the sensor unit 44A includes a housing 88, a transmitter 90, a receiver 92, a sensor 94 and control components or circuitry 96 (hereinafter referred to as “control circuitry”). The receiver 92 and the control circuitry 96 receive wireless signals generated by one or more of the interrogators 40, capture energy from the received wireless signals and use the captured energy to energize the transmitter 90 and the sensor 94.

The sensor 94 is operable to sense a condition within the storage area 42 and communicate the sensed data to the controller 96 and/or the transmitter 90 to be communicated to one or more of the interrogators 40 via a wireless signal. The sensor 94 may include, for example, a temperature sensor, a humidity sensor, a chemical sensor, a biosensor (for example, a mold sensor), or an acoustic sensor (for example, a piezoelectric sensor). The biosensor and/or the acoustic sensor may be used to detect the presence of pests, such as insects or rodents, in the storage area 42. The sensor 94 is powered by energy derived from wireless signals communicated by the interrogators 40 as explained above, and thus may generate sensor data only when energized by the interrogator signal. The sensor 94 may be entirely encased in the housing 88 or may be partially or entirely external to the housing 88.

The control circuitry 96 controls operation of the sensor unit 44A and enables communication between components of the sensor unit 44A. The control circuitry 96 may be as simple as a few discrete components or may be more complex. In some embodiments, the control circuitry 96 includes one or more digital processors.

The transmitter 90 generates a wireless signal, such as an RF signal, that is formatted for compatibility with the interrogators 40. The transmitter 90 may be similar or identical to wireless transmitters used in RFID tags, as explained above. The transmitter 90 communicates sensor data in the wireless signal so that the sensor data can be received and used by the interrogators 40 and/or the computing system 38. The transmitter 90 of each of the sensor units 44A may have similar or identical operational characteristics to facilitate triangulation or other operations.

A sensor unit 44B constructed according to another exemplary embodiment is illustrated in FIG. 10B. The sensor unit 44B is similar to the sensor unit 44A, described above, except that the sensor unit 44B includes two sensors 94A, 94B and a memory/storage component 98. The first sensor 94A may be identical to the sensor 94, discussed above. The second sensor 94B may be similar to the first sensor 94A but may be configured to sense another condition other than the condition sensed by the first sensor 94A. The first sensor 94A may sense temperature, for example, while the second sensor 94B may sense humidity. Alternatively, both sensors 94A, 94B may be configured to sense the same condition to, for example, provide redundancy.

The memory/storage component 98 is operable to store or retain data, such as sensor data generated by one of the sensors 94A, 94B. The sensor unit 44B is passive and therefore is energized by power derived from wireless signals transmitted by the interrogators, so the memory/storage component 98 may include non-volatile data storage to retain data when the sensor unit 44B is not energized. Thus, when the sensor unit 44B is energized, the control circuitry 96 may be configured to store sensor data in the memory/storage component 98 in addition to communicating the data in a wireless signal via the transmitter 90.

The control circuitry 96 may be configured to communicate data from one or both sensors 94A, 94B to the interrogators 40 via signals transmitted from the wireless transmitter 90. The control circuitry 96 may be configured to determine, from an interrogator signal received via the receiver 92, which one of the two sensors 94A, 94B to collect data from to include in the wireless signal communicated to the interrogators 40. The control circuitry 96 may further be configured to store or retrieve data from the memory/storage component 98 in response to instructions received from one of the interrogators 40.

A sensor unit 44C constructed according to another exemplary embodiment is illustrated in FIG. 10C. The sensor unit 44C is similar to the sensor unit 44A, described above, except that the sensor unit 44C does not include a sensor. This embodiment may be used to estimate a collective profile of the particulate material in the storage area 42, including a collective shape of the particulate material and/or a total amount of particulate material.

A sensor unit 44D constructed according to another exemplary embodiment is illustrated in FIG. 10D. The sensor unit 44D is similar to the sensor unit 44A, described above, except that the sensor unit 44D is an active unit, meaning that it includes a power source 100 and does not depend on energy from an external wireless signal for power. Because the sensor unit 44D has its own power source, it does not need a receiver for receiving signals from the interrogators, thus the sensor unit does not include a receiver. In this embodiment, the sensor unit 44D may automatically and periodically take sensor samples and communicate sensor data to the interrogators 40 via the transmitter 90. The power source 100 may be a battery and, in some embodiments, may include a replaceable battery. It will be appreciated that even though the sensor unit 44D does not require a receiver for generating power, it may still include a receiver to enable two-way communications with the interrogators.

A sensor unit 44E constructed according to another exemplary embodiment is illustrated in FIG. 10E. The sensor unit 44E is similar to the sensor unit 44D, described above, except that the sensor unit 44E includes a memory/storage component 98 and a receiver 102. As explained above, the memory/storage component 98 is operable to store or retain data generated by the sensor 94. The receiver 102 may be used to receive signals from the interrogators 40 to enable the interrogators 40 to poll the sensor units 44 for data.

The sensor unit housing 88 encases the other components of the sensor unit 44 and is sufficiently durable to protect the other components from damage due to compression, physical movement, shock, moisture, dust and other hazards characteristic of agriculture and manufacturing environments. The housing 88 also allows the sensor units 44 to detect conditions outside the housing 88. As explained above, this may require some or all of the sensor or sensors 94 to be located outside the housing 88.

The housing 88 may be of a shape and/or texture to allow the sensor units 44 to be distributed through the particulate material through a natural and random mixing process. A natural and random mixing process is one in which the sensor units 44 mix among the particulate material in the same manner as other particulate material. If the particulate material is grain, for example, the housing 88 may present the same size, shape and/or texture (on an outer surface) as individual kernels of the grain.

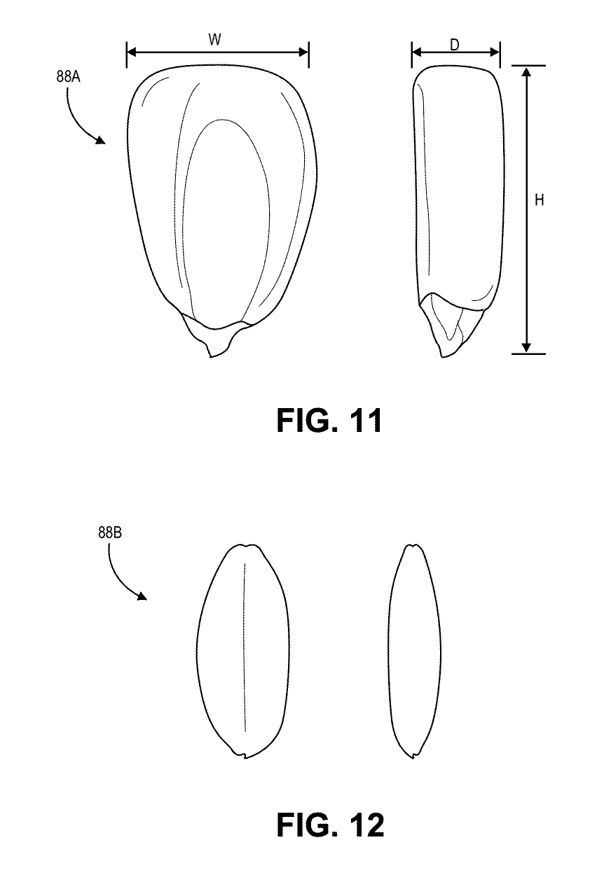

An exemplary sensor unit housing 88A is illustrated in FIG. 11, wherein the size and shape of the housing 88A approximates the size and shape of a kernel of corn. The width W may be between about 1.0 mm and about 10.0 mm, the depth D may be between about 0.1 mm and about 5.0 mm, and the height H may be between about 2.0 mm and about 15.0 mm. The weight of the sensor unit may approximate that of a kernel of corn and may be, for example, between about 0.1 g and about 5.0 g and may particularly be between 0.25 g and 0.30 g. Another exemplary sensor unit housing 88b is illustrated in FIG. 12, wherein the housing presents the size and shape of a kernel of wheat. The width may be between about 0.5 mm and about 2.0 mm, the depth may be between about 0.1 mm and about 0.5 mm, and the height may be between about 0.5 mm and about 5.0 mm. The weight of the sensor unit may approximate that of a kernel of wheat and may be, for example, between about 0.01 g and about 2.0 g. These are but two examples, the housing may present the size and shape of virtually any grain or other particulate material including, for example, beans, milo, barley, rice and so forth.

Furthermore, in some embodiments the sensor units 44 do not present the same size and shape of the particulate material, but may present a spherical or other shape. Regardless of the shape, the sensor units 44 may be between about 1.0 mm and about 50 mm in length or diameter, more particularly between about 2.0 mm and about 20 mm in length or diameter, more particularly between about 5.0 mm and about 15 mm.

One or more components of the sensor unit 44 may have magnetically-responsive properties to facilitate removal of the sensor unit 44 from the particulate material. The housing 88, for example, may be magnetically responsive such that when exposed to a magnetic field, the magnetic field results in mechanical force on the sensor unit 44 in a direction, for example, toward or away from the source of the magnetic field. The resulting force may be sufficient to separate the sensor unit 44 from the particulate material and may be, for example, equal to or greater than the force of gravity on the sensor unit 44 when exposed to a magnetic field within the range of from about 0.001 Tesla to about 1.0 Tesla or, more particularly, within the range of from about 0.01 Tesla to about 0.1 Tesla. The mechanical force exerted on the sensor unit 44 may be two, three, four, five, ten, fifteen or twenty times the force of gravity on the sensor unit 44 when exposed to a magnetic field within the range of from about 0.001 Tesla to about 1.0 Tesla or, more particularly, within the range of from about 0.01 Tesla to about 0.1 Tesla.

The magnetically-responsive properties may be leveraged to separate the sensor units 44 from the particulate material by, for example, exposing the particulate material to a magnetic field as the material passes through a conveyor or other chokepoint wherein the magnetic forces applied on the sensor units 44 cause them to follow a different path than the particulate material or otherwise separate therefrom. Other methods may be used for separation, including making the sensor units 44 larger than the particulate material such that the particulate material passes through a filter while the sensor units 44 are caught by the filer (or vice versa).

The sensor units 44 may be mixed approximately evenly among the particulate material or may be distributed through the storage area 42 and/or the particulate material according to other methods, depending on such factors as the material and the conditions monitored. The ratio of sensor units 44 to particulate material may be one sensor unit per 100, 500, 1,000, 10,000 or 100,000 particulates, or 0.01, 0.1, 1, 10, 100 or 1,000 sensor units per cubic foot of space in the storage area 42. The particular number or density of sensor units 44 is not critical to the present invention and variations beyond the ranges mentioned in this paragraph are within the ambit of the invention.

The computing system 38 uses information and data from the interrogators to assess and monitor conditions inside the storage area 42 (including inside a mass of particulate material) and to respond to exceptional conditions inside the storage area 42. Additionally or alternatively, the computing system 38 may generate notices, alerts and/or reports relating to the sensed conditions and store sensed conditions or other information in a database. The computing system 38 may respond to the exceptional conditions by communicating an alert or warning to one or more persons or entities, by communicating control instructions to a machine or system, or both. In some embodiments, the computing system 38 determines a location of each of the sensor units 44 and associates data from each sensor unit 44 with the location of the respective sensor. The computing system 38 may also use spatial data interpolation techniques to estimate condition values at locations other than the locations of sensed values.

The computing system 38 communicates with the interrogators 40 via a direct wired or wireless connection or via one or more wired or wireless networks, including a local area network and/or the Internet. The computing system 38 may include one or more of the computing devices 12-32 illustrated in FIG. 1 and described above. By way of example, certain functions of the computing system described herein may be implemented by an Internet- or cloud-based computer or system including one of the computing devices 12, 14, 16. Furthermore, the functionality may be accessible via one of the device 18-32. A computer such as the laptop computer 28 or the desktop computers 30, 32 may include software configured to allow a user to view and manipulate a two- or three-dimensional model or representation of a storage area including one or more indicia of sensor data and sensor location. A user may further be able to view information via a handheld or wearable device like the devices 18-24 or on a machine display that is associated with the device or system 26. The computing system 38 may comprise a single computer, such as a laptop or desktop computer 28-32 or a handheld device 20-24 in direction communication with the interrogators 40.

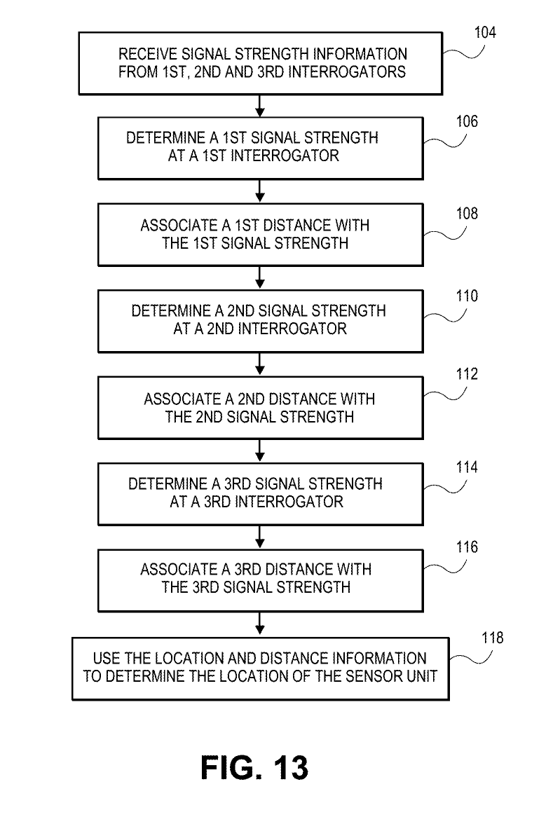

The computing system 38 determines positions of the sensor units 44 using, for example, principles of triangulation. Various steps involved in an exemplary process of determining locations of the sensor units 44 in the storage area 42 is illustrated in the flow diagram of FIG. 13. The process involves associating each of the interrogators 40 with a location relative to the particulate storage area 42. A three-dimensional virtual model of the storage area may be constructed, for example, and each of the interrogators 40 may be associated with a location in the model. Each of the interrogators 40 is operable to communicate data or information, such as sensor data, to the computing system 38 either directly or indirectly via any of various communications means, as explained above. Each interrogator 40 may be configured to communicate a unique identifier, such as an alpha-numeric identifier, to the computing system 38 to enable the computing system 38 to associate the data or information received from the interrogator 40 with the location of the interrogator 40.

This method assumes the signal strength is associated with a distance, and the association may be determined beforehand by, for example, running tests in the actual environment. Alternatively or additionally, sensor unit transmitter specifications may be used to determine or estimate signal strength versus distance from transmitter. The signal strength may also be influenced by characteristics of the signal transmitted by the interrogators 40, including the number and density of the interrogators.

First, signal strength information is received from first, second and third interrogators 40, as depicted in block 104. Each of the interrogators 40 may be configured to generate a numeric value related to the signal strength, for example, and communicate the numeric value to the computing system 38. Then, the computing system 38 determines a first signal strength at a first interrogator 40, as depicted in block 106. This may involve simply associating the received numeric value with the location of the interrogator 40, or may involve manipulating the numeric value. The computing system 38 then associates a first distance with the first signal strength, as depicted in block 108. This step may involve using a look-up table to match the first signal strength with a distance, or applying a mathematical equation to the first signal strength. The resulting distance may be expressed, for example, in centimeters or meters, such as 20 cm, 70 cm, 0.5 m, 2.7 m or 6.8 m.

In the same manner as discussed above, the computing system determines a second signal strength at a second interrogator, as depicted in block 110, associates a second distance with the second signal strength, as depicted in block 112, determines a third signal strength at a third interrogator, as depicted in block 114, and associates a third distance with the third signal strength, as depicted in block 116. At this point the computing system 38 has a distance associated with each of three known locations (that is, the location of each of the interrogators). The computing system 38 then uses the location of each of the three interrogators and the distance associated with each of the interrogators to determine the location of the sensor unit, as depicted in block 118. More specifically, and by way of example, the computing system 38 identifies a point in space that is separated from the location of the first interrogator 40 by the first distance, is separated from the location of the second interrogator 40 by the second distance and is separated from the location of the third interrogator 40 by the third distance. That point in space corresponds to the location or estimated location of the sensor unit 44 that generated the sensor signal.

Principles of triangulation are well-known in the art and the process illustrated in FIG. 13 is one example of how triangulation may be implemented. Variations may be used without departing from the spirit or scope of the invention, including using signal strength information from additional interrogators.

Once the computing system 38 has determined the location of each of the sensor units, it associates the sensor data originating from each of the sensor units 44 with the location of the respective sensor unit 44. Thus, the computing system 38 is operable to create a three-dimensional virtual model of the particulate storage area and assign condition values to different portions of the model. If the sensed condition is temperature, for example, the computing system 38 is operable to assign a temperature value to each of various portions or locations of the model.

The computing system 38 may use spatial data interpolation techniques to estimate condition values at locations other than the locations of the sensor units. Using these techniques, the computing system 38 may estimate condition values through the entire particulate storage area and use both the sensed and the estimated condition values as explained herein. Spatial data interpolation involves estimating one or more variables at one or more unobserved locations in geographic space based on values at observed locations. If the sensed condition is temperature, the sensor units 44 would generate a measured temperature value for each location associated with a sensor unit 44. Using spatial data interpolation, the computing system 38 would estimate temperature values at other locations in the storage area 42, potentially assigning a measured or estimated temperature value to each location in the storage area 42.

The computing system 38 may perform the spatial data interpolation using inverse distance weighting, which involves attenuating the estimated variable with decreasing proximity to the observed location. The following equation is an example of an inverse distance weighting function that may be used to find an interpolated value u at a given point x based on samples uᵢ=u(xᵢ) for i=0, 1, . . . , N:

$$u ( x ) = {\sum }_{i = 0}^{N} \frac{{w}_{i} ( x ) {u}_{i}}{{\sum }_{j = 0}^{N} {w}_{j} ( x )}$$

$$where$$

$${w}_{i} ( x ) = \frac{1}{{d ( x , {x}_{i} )}^{p}}$$

$$d = the distance from the kn own point {x}_{i}$$

$$p = a positive real number called the “ power parameter ”$$

This is but one example, and other spatial interpolation methods may be used, including the “Kriging” or “Gaussian” method and the polynomial spline method.

The computing system 38 may detect an exceptional condition and respond to the exceptional condition by, for example, generating a warning or alert, by communicating control instructions to a machine or system, or both. An exceptional condition may related to any of the sensed conditions mentioned previously, and may be defined by a user. Thus, an exceptional condition may be virtually any condition defined by a user and may be correspond simply to any condition (measured or estimated) under which the computing system 38 generates a response. Exceptional conditions may include a temperature value exceeding a predetermined threshold, a humidity value exceeding a predetermined threshold, a temperature differential within the particulate storage area exceeding a predetermined threshold, or a humidity differential within the particulate storage area exceeding a predetermined threshold. Exceptional conditions may also involve the collective profile of the particulate material in the storage area 42, such as a total amount of the particulate material, a collective shape of the particulate material, or both. These are but a few examples.

In some embodiments, the computing system 38 may use information or data relating to conditions external to the storage area 42 to identify exceptional conditions and to determine an appropriate response to an exceptional condition. By way of example, the computing system 38 may receive ambient condition data from the sensor 48 (FIG. 3) and use that data to compare external conditions with the conditions inside the storage area 42 determined via the sensor units 44. In one implementation, the computing system 38 compares a temperature (e.g., an average temperature) inside the storage area 42 with an temperature outside of the storage area to identify an internal/external temperature differential. Such temperature differentials may lead to moisture problems with the particulate material if particulate material near the edges of the storage area 42 experience a dramatic change in temperature that would cause convention currents to occur in the particulate material wherein moisture from warmer portion of the storage area 42 is picked up in the air and then condenses out of the air in cooler portions of the storage area 42. Such isolation of moisture may result in damage to the particulate material where the moisture levels increase.

In this implementation, the external temperature may be measured by a sensor in direct or indirect communication with the computing system 38, such as the sensor 48 illustrated in FIG. 3. Alternatively, the external temperature may be determined via other means, including using weather information available via the Internet. The computing system 38 may generate an alert and/or activate a device or system, such as an aeration system, if the internal/external temperature differential exceeds a predetermined level, such as, for example, 5° C., 10° C., 15° C. or 20° C. The computing system 38 may also use external condition data or information to determine an appropriate response to an exceptional condition. By way of example, the computing system 38 may use the sensor 48 and/or information available from the Internet to determine an external humidity level and/or to determine whether precipitation conditions exist prior to activating an aeration system that may be affected by humidity or precipitation.

The computing system 38 may generate one or more alerts or notices in any of various forms. The computing system 38 may generate an alert that is presented to a user on a display of one or more of the computing devices 18-24 or 28-30, for example, in the form of a text message, email message, social media notification, or a notification generated by a user interface generated by software that is native to the device. The computing system 38 may also communicate control instructions to a machine or system in response to detecting an exceptional condition. The computing system 38 may communicate control instructions to an aeration system associated with the storage bin 50, for example, to activate the aeration system if a temperature or humidity differential inside the storage area 42 exceeds a predetermined level.

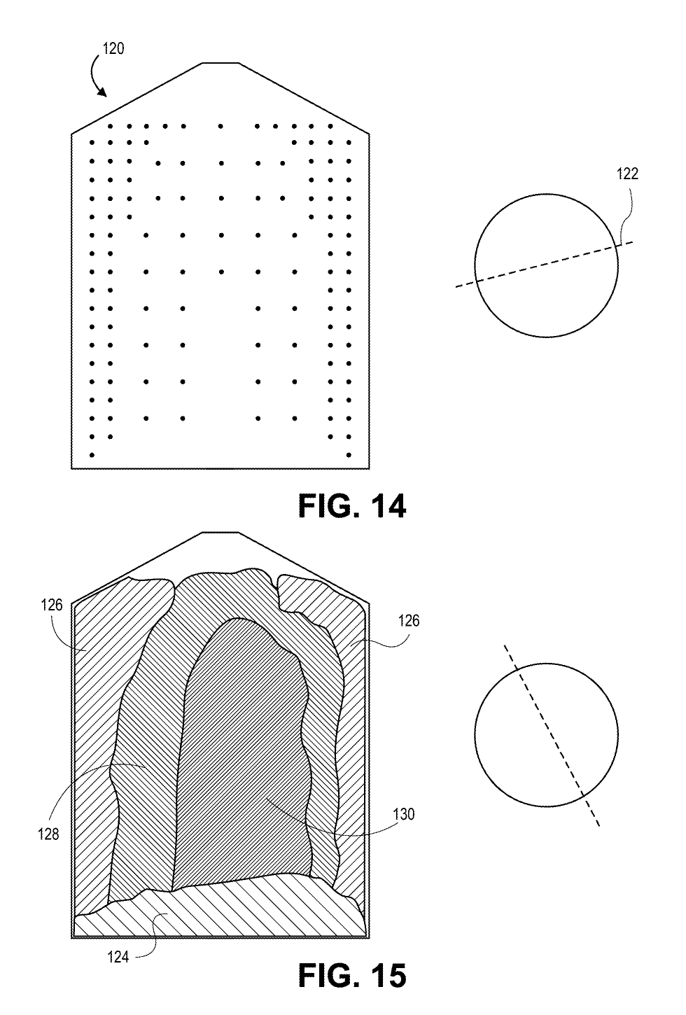

The computing system 38 may generate a graphic representation of the storage area 42 along with indicia of the sensed and measured condition values associated with the storage area 42. Such a graphic representation would allow users to quickly and easily view conditions of interest in the storage area 42. The graphic representation could include one or more two-dimensional and/or three-dimensional views of the particulate storage area. Various exemplary two-dimensional graphic representations of the storage area are illustrated in FIGS. 14-17. In FIG. 14, a depiction 120 of a cross-section of a storage bin is illustrated wherein density of dots indicates intensity or magnitude of a particular condition. More densely-situation dots may indicate a higher temperature or a higher humidity, for example, while the less densely-situated dots may indicate a lower temperature or a lower humidity. Rather than dots the graphic may include a continuous heat map, where different colors represent different condition intensities or magnitudes.

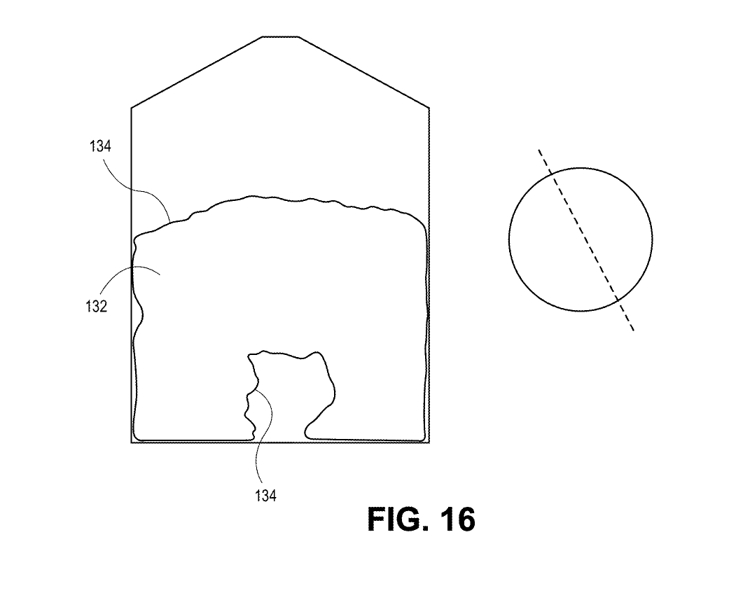

A view line 122 may indicate which portion of the storage area 42 is depicted by defining a cross section of the storage area 42. As illustrated, the cross-section 120 of the storage bin corresponds to a side elevation view of the bin, while the view of the view line 122 corresponds to a plan view of the storage bin. Other configurations may be used. A user may change the cross section of the storage area 42 depicted by moving the view line 122. A similar two-dimensional graphic is illustrated in FIG. 15, wherein various continuous regions 124, 126, 128, 130 are depicted with each region corresponding to a range of condition values. FIG. 16 illustrates a similar two-dimensional graphic presenting a representation of a collective profile of the particulate material 132 in the storage area 42, including a collective shape of the particulate material indicated by an outline 134 of the material.

Although not depicted in the figures, the two-dimensional graphic representation of storage area 42 may be as simple as an outline depicting the shape of the area along with numbers placed at various locations in the outline, the numbers indicating measured and/or estimated condition values. The location of the numbers indicating the location of the condition value and the number indicating a magnitude of the condition value.

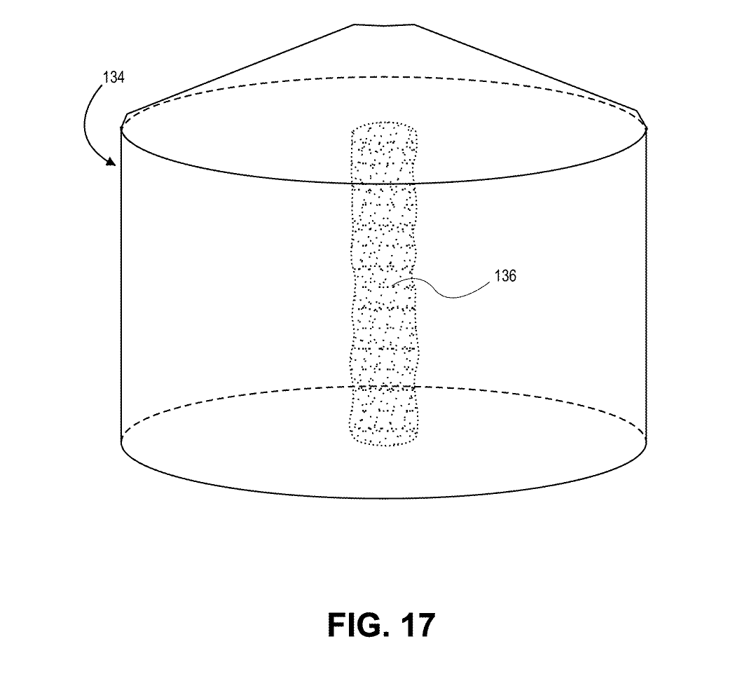

The computing system 38 may further be configured to generate a three-dimensional representation 134 of the storage area with indicia depicting one or more conditions inside the storage area, as illustrated in FIG. 17. The representation 134 illustrates a column of space 136 inside the storage area 42 where an exceptional condition, such as a moisture level, has been detected. The computing system 38 may be configured to enable one or more users to view and manipulate the three-dimensional representation 134 by, for example, enlarging or “zooming in on” certain aspects of the representation or by rotating the representation.

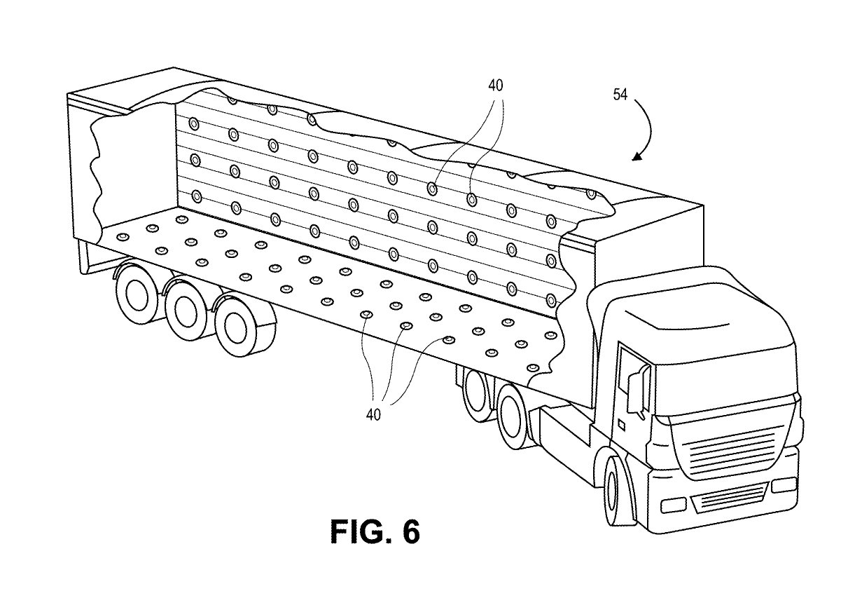

In a first exemplary scenario, the system 36 (or 46) is used to assess and monitor grain conditions when the grain is harvested, stored and/or transported. The grain may be harvested using a combine harvester and transferred to a grain truck (e.g., similar to the truck 54 illustrated in FIG. 6) or a grain wagon for transport to a grain storage facility, such as a grain bin or series of grain bins similar to the bins 50 or 52. The sensor units 44 may be mixed with the grain at the time of harvest or when the grain is transferred from the harvester to the truck or wagon. The truck or wagon may correspond to a first storage area 42 and include interrogators 40 as explained above for communicating with the sensor units 44 to determine one or more conditions, such as temperature and humidity inside the storage area 42. If the computing system 38 identifies an exceptional condition it may generate an alert to a user who may, for example, take action to transfer the grain from the truck or the wagon or move the truck or wagon to a sheltered area.

The grain may be transferred to a grain storage bin that is also equipped with interrogators configured as explained herein for further monitoring of conditions inside the storage area. As the computing system 38 monitors conditions in the grain bin, it may generate reports and alerts, and may activate devices or systems (e.g., aeration systems) to preserve optimal storage conditions. The computing system 38 may also be used to determine a fill level of a particular storage area 42, such as a grain bin, to determine whether additional grain may be added to the storage area and, if so, how much.

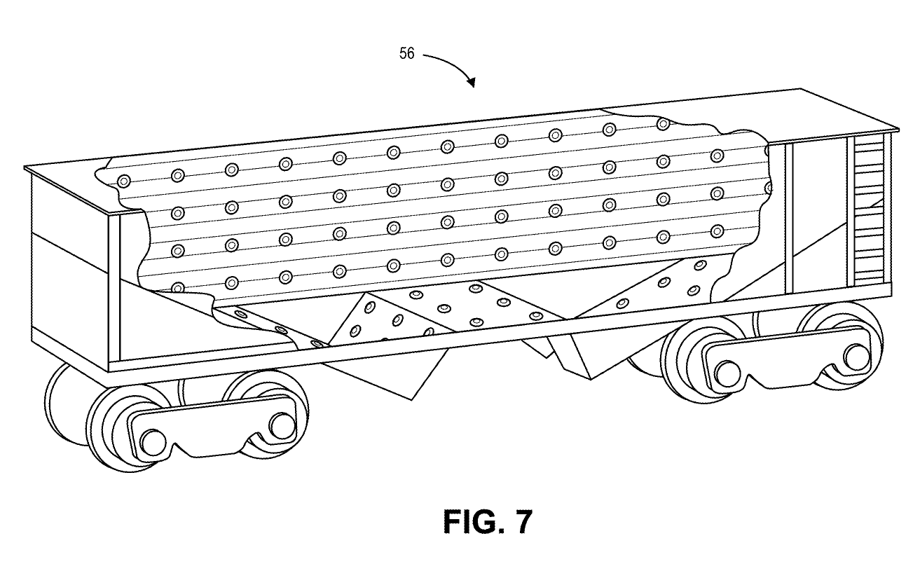

The computing system 38 may continue to monitor and track the grain as it is transferred and shipped, including if the grain is shipped via a railroad hopper car, such as a car similar to the car 56 illustrated in FIG. 7. The computing system 38 may record information relating to each container, the time period when the grain is stored in each container, and the conditions monitored while the grain is stored in each container. The computing system 38 may retain that information and generate a report including all of that information at any point in the storage and transfer process as evidence, for example, that the grain was subject to proper storage and transfer conditions for optimum quality.

In another exemplary scenario, the system 36 (or 46) is used to assess and monitor one or more conditions of a manufactured particulate material, such as fertilizer or animal feed pellets. The sensor units 44 may be mixed with the manufactured particulate material at virtually any point during or after the manufacturing process. If certain temperature levels are required or potentially detrimental to the manufacturing process, for example, the sensor units 44 may be used to monitor temperature at various stages of the manufacturing process and/or after the manufacturing process. Collection bins or hoppers used in the manufacturing process may correspond to storage areas 42 wherein the interrogators 40 may be associated with the collection bins or hoppers. The computing system 38 could then collect and assess condition information at various points through the manufacturing process, as well as condition information associated with long-term storage and/or transportation of the manufactured particulate material after the manufacturing process.

Although the invention has been described with reference to the preferred embodiment illustrated in the attached drawing figures, it is noted that equivalents may be employed and substitutions made herein without departing from the scope of the invention as recited in the claims. The computing system, for example, may include processors or other components or devices in or associated with the wireless interrogators such that the wireless interrogators perform some of the functions associated herein with the computing system, such as determining a location of each of the sensor units. Furthermore, while a method of triangulation has been described herein for determining locations of the sensor units, other methods may be used, including estimating locations of sensor units based on proximity to a single interrogator.

Having thus described the preferred embodiment of the invention, what is claimed as new and desired to be protected by Letters Patent includes the following:

1. A computing system comprising:
one or more computing devices; and
a non-transitory computer-readable medium communicatively coupled with the one or more computing devices and having instructions stored thereon which, when executed by the one or more computing devices, cause the one or more computing devices to perform operations comprising—
receiving information about a plurality of sensor unit signals, the information including a signal strength associated with each signal at each of a plurality of different locations and sensor data communicated in each of the signals,
determining a location of each of the sensor units using the signal strength information, and
associating sensor data from each sensor unit with the location of the corresponding sensor unit.

2. The computing system as set forth in claim 1, the operations further comprising generating a graphic representation of an area associated with the sensor units and presenting the graphic representation to a user, the graphic representation including an indicia of the sensor data associated with each of the sensor unit signals and an indicia of the location of the sensor unit associated with each of the sensor unit signals.

3. The computing system as set forth in claim 2, the graphic representation of the area including a three-dimensional model of the area, a graphic representation of the location of each of the sensor units in the model, and a graphic representation of the sensor data associated with each of the sensor units.

4. The computing system as set forth in claim 2, the operations further comprising—
receiving an input from a user, and
in response to the input, modifying the graphic representation of the area.

5. The computing system as set forth in claim 1, the operations further comprising—
using the sensor data from at least one of the plurality of signals to estimate values at locations other than the locations of the sensor units.

6. The computing system as set forth in claim 1, the operations further comprising—
determining when the sensor data indicates an exceptional condition, and
communicating an alert to a user indicating the presence of the exceptional condition.

7. The computing system as set forth in claim 1, the operations further comprising—
determining when the sensor data indicates an exceptional condition, and
communicating instructions to a machine or system to respond to the exceptional condition.

8. The computing system as set forth in claim 1, the operations further comprising—
receiving a container identifier associated with a container from which the signals originate,
storing sensor information and the identifier in a database, and
associating the sensor information with the identifier.

9. The computing system as set forth in claim 8, the operations further comprising using sensor information and at least two container identifiers from the database to generate a report including information about conditions in multiple containers.

10. A non-transitory computer-readable medium having instructions stored thereon which, when executed by one or more computing devices, cause the one or more computing devices to perform operations comprising—
receiving information about a plurality of sensor unit signals, the information including a signal strength associated with each signal at each of a plurality of different locations and sensor data communicated in each of the signals,
determining a location of each of the sensor units relative to the different locations using the signal strength information, and
associating sensor data from each sensor unit with the location of the sensor unit.

11. The non-transitory computer-readable medium as set forth in claim 10, the operations further comprising generating a graphic representation of an area associated with the sensor units and presenting the graphic representation to a user, the graphic representation including an indicia of the sensor data associated with each of the sensor unit signals and an indicia of the location of the sensor unit associated with each of the sensor unit signals.

12. The non-transitory computer-readable medium as set forth in claim 11, the graphic representation of the area including a three-dimensional model of the area, a graphic representation of the location of each of the sensor units in the model, and a graphic representation of the sensor data associated with each of the sensor units.

13. The non-transitory computer-readable medium as set forth in claim 11, the operations further comprising—
receiving an input from a user, and
in response to the input, modifying the graphic representation of the area.

14. The non-transitory computer-readable medium as set forth in claim 10, the operations further comprising—
using the sensor data from at least one of the plurality of signals to estimate values at locations other than the locations of the sensor units.

15. The non-transitory computer-readable medium as set forth in claim 10, the operations further comprising—
determining when the sensor data indicates an exceptional condition, and
communicating an alert to a user indicating the presence of the exceptional condition.

16. The non-transitory computer-readable medium as set forth in claim 10, the operations further comprising—
receiving a container identifier associated with a container from which the signals originate,
storing sensor information and the identifier in a database, and
associating the sensor information with the identifier.

17. A computing system comprising:
one or more computing devices; and
a non-transitory computer-readable medium communicatively coupled with the one or more computing devices and having instructions stored thereon which, when executed by the one or more computing devices, cause the one or more computing devices to perform operations comprising—
receiving information about a plurality of sensor unit signals, the information including a signal strength associated with each signal at each of a plurality of different locations and sensor data communicated in each of the signals, the sensor data relating to an ambient condition,
determining a location of each of the sensor units relative to the different locations using the signal strength information,
associating sensor data from each sensor unit with the location of the corresponding sensor unit,
using the sensor data from at least one of the plurality of signals to estimate ambient condition values at locations other than the locations of the sensor units, and
generating a graphic representation of an area associated with the sensor units and presenting the graphic representation to a user, the graphic representation including indicia of the sensor data associated with each of the sensor unit signals, of the location of the sensor unit associated with each of the sensor unit signals, of the estimated condition values, and of the location associated with each of the estimated condition values.

18. The computing system as set forth in claim 17, the operations further comprising—
determining when the sensor data or the estimated condition values indicate an exceptional condition, and
communicating an alert to a user indicating the presence of the exceptional condition.

19. The computing system as set forth in claim 17, the operations further comprising—
determining when the sensor data or the estimated condition values indicate an exceptional condition, and
activating or deactivating operation of a machine in response to determining the presence of the exceptional condition.

20. A computing system comprising:
one or more computing devices; and
a non-transitory computer-readable medium communicatively coupled with the one or more computing devices and having instructions stored thereon which, when executed by the one or more computing devices, cause the one or more computing devices to perform operations comprising—
receiving information about a plurality of sensor unit signals, the information including a signal strength associated with each signal at each of a plurality of different locations,
determining a location of each of the sensor units using the signal strength information, and
estimating an amount of particulate material associated with the sensor units using the location of each of the sensor units.

21. The computing system as set forth in claim 20, the operations further comprising generating a notification to a user if the amount of particulate material exceeds a predetermined amount.

22. The computing system as set forth in claim 20, the operations further comprising estimating a collective shape of the particulate material using the location of each of the sensor units.

23. The computing system as set forth in claim 20, the operations further comprising generating a notification to a user if the collective shape of the particulate material corresponds to a predetermined shape.
